(19) United States
(12) Patent Application Publication
(10) Pub. No.: US 2026/0369388 A1
(43) Pub. Date: Nov. 19, 2026

(54) DIAGNOSIS SUPPORT SYSTEM, DIAGNOSIS SUPPORT METHOD, AND NON-TRANSITORY COMPUTER-READABLE MEDIUM

(71) Applicant: NEC Corporation, Tokyo (JP)

(72) Inventors: Osamu HISAMATSU, Tokyo (JP); Mitsuru NOMA, Tokyo (JP); Keisuke SUZUKI, Tokyo (JP)

(73) Assignee: NEC Corporation, Tokyo (JP)

(21) Appl. No.: 19/659,204

(22) Filed: Apr. 27, 2026

(30) Foreign Application Priority Data
May 15, 2025 (JP) 2025-081902

(51) Int. Cl. G16H 30/40; G16H 50/20

(52) U.S. Cl. CPC G16H 30/40; G16H 50/20

(57) ABSTRACT

A diagnosis support system according to the present disclosure is a diagnosis support system that supports a medical diagnosis for a user, and includes at least one memory storing instructions and at least one processor configured to execute the instructions to acquire an original image including signal waveforms of a plurality of types of biological signals for the same user, and adjust at least one of scales of the plurality of signal waveforms in the original image based on a predetermined adjustment criterion for optimizing the at least one scale.

### INCORPORATION BY REFERENCE

This application is based upon and claims the benefit of priority from Japanese patent application No. 2025-081902, filed on May 15, 2025, the disclosure of which is incorporated herein in its entirety by reference.

### TECHNICAL FIELD

The present disclosure relates to a diagnosis support system, a diagnosis support method, and a non-transitory computer readable medium.

### BACKGROUND ART

Japanese Unexamined Patent Application Publication No.2021-112479 discloses a technique for diagnosing a predetermined disease. An electrocardiographic signal analyzing apparatus according to Japanese Unexamined Patent Application Publication No.2021-112479 generates a one heartbeat image by normalizing an electrocardiographic signal and aligning heartbeat intervals. The electrocardiographic signal analyzing apparatus determines whether the patient has aortic stenosis using the one heartbeat image.

### SUMMARY

There is a need to diagnose various diseases by utilizing biological signals such as an electrocardiographic signal. Japanese Unexamined Patent Application Publication No.2021-112479 discloses a technique for using an electrocardiographic signal by an electrocardiograph for diagnosis of aortic stenosis, but does not disclose a technique for using various other biological signals.

The present disclosure has been made to solve such a problem, and an example object of the present disclosure is to provide a diagnosis support system, a diagnosis support method, and a non-transitory computer-readable medium capable of supporting diagnosis in which various biological signals are integrated.

A diagnosis support system according to an example aspect of the present disclosure is a diagnosis support system that supports a medical diagnosis for a user, the diagnosis support system including at least one memory storing instructions and at least one processor configured to execute the instructions to acquire an original image including signal waveforms of a plurality of types of biological signals for the same user, and adjust at least one of scales of the plurality of signal waveforms in the original image based on a predetermined adjustment criterion.

A diagnosis support method according to an example aspect of the present disclosure is a diagnosis support method in which a computer supports a medical diagnosis for a user, the diagnosis support method including acquiring an original image including signal waveforms of a plurality of types of biological signals for the same user, and adjusting at least one of scales of the plurality of signal waveforms in the original image based on a predetermined adjustment criterion.

A non-transitory computer-readable medium according to an example aspect of the present disclosure stores a program for supporting medical diagnosis for a user, the program causing a computer to execute acquiring an original image including signal waveforms of a plurality of types of biological signals for the same user, and adjusting at least one of scales of a plurality of the signal waveforms in the original image based on a predetermined adjustment criterion.

The present disclosure can provide a diagnosis support system, a diagnosis support method, and a non-transitory computer-readable medium capable of supporting diagnosis of various diseases.

### BRIEF DESCRIPTION OF DRAWINGS

The above and other aspects, features, and advantages of the present disclosure will become more apparent from the following description of certain example embodiments, taken in conjunction with the accompanying drawings, in which:

FIG. 1 is a block diagram illustrating a configuration of a diagnosis support system according to the present disclosure;

FIG. 2 is a flowchart illustrating a flow of a diagnosis support method by a diagnosis support system;

FIG. 3 is a block diagram illustrating a configuration of another diagnosis support system according to the present disclosure;

FIG. 4 is a flowchart illustrating an example of a flow of a preprocessing method for an original image;

FIG. 5 is a flowchart illustrating an example of a flow of a method of adjusting a scale of a signal waveform; and

FIG. 6 is a diagram illustrating a hardware configuration example of a diagnosis support system.

### EXAMPLE EMBODIMENT

Hereinafter, example embodiments according to the present disclosure will be described in detail with reference to the drawings. In the drawings, the same or related elements are denoted by the same reference signs. Furthermore, for clarity of description, redundant description will be appropriately omitted.

### First Example Embodiment

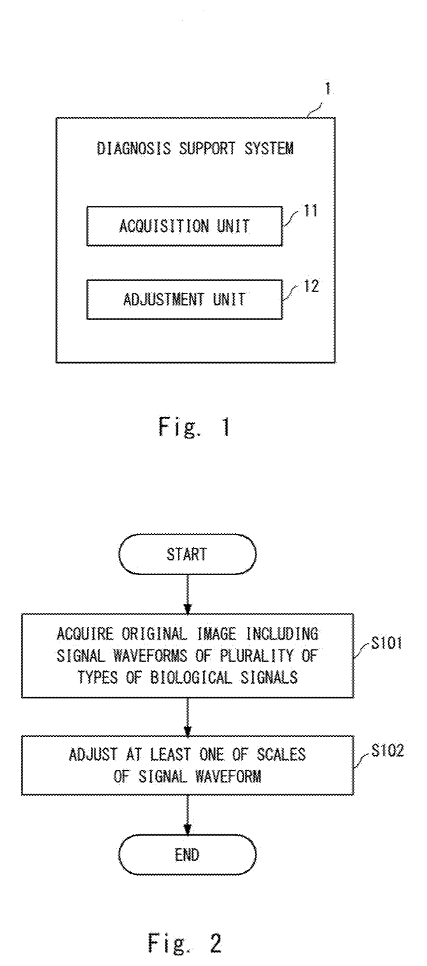

A first example embodiment according to the present disclosure will be described below with reference to the drawings. FIG. 1 is a block diagram illustrating a configuration of a diagnosis support system 1 according to the present disclosure. The diagnosis support system 1 is typically one or two or more computer apparatuses that operate in a case where a processor executes a program stored in a memory. In other words, the diagnosis support system 1 may be configured by one computer apparatus or may be configured by two or more computer apparatuses. For example, the diagnosis support system 1 is one or two or more server apparatuses.

The diagnosis support system 1 is an information processing system that supports medical diagnosis for a user. Specifically, the diagnosis support system 1 is an information processing system for adjusting at least one of scales of signal waveforms included in original images of a plurality of types of biological signals for the same user based on a predetermined adjustment criterion. Here, the “user” may or may not be a patient in a predetermined medical institution. That is, the “user” may or may not be a person who is already suffering from some kind of disease. Furthermore, “medical diagnosis” refers to determining the health condition of the user, the presence or absence of a disease, and the type of a disease. The diagnosis support system 1 may, for example, support medical diagnosis by a doctor, or may support diagnosis by a predetermined computer system (not illustrated) that performs a medical diagnosis.

The “biological signal” refers to a signal that temporally captures an electrical, physical, or chemical change generated from the inside, the surface, or the like of a living body (user). The biological signal includes, for example, an electrocardiogram, an electroencephalogram, an electromyogram, a pulse, a blood pressure, and respiration, but is not limited thereto. In addition, the biological signal may include a body temperature. Furthermore, the “signal waveform” expresses time-series information observed for a biological signal generated by a living body as a waveform. The signal waveform is time-series information of the biological signal in a predetermined period. The signal waveform typically includes a value of a biological signal related to a time or a date on a time axis. The signal waveform is, for example, a graph in which time-series data is plotted with a horizontal axis as a time axis and a vertical axis as a biological signal axis. Here, the biological signal axis is an axis indicating the value of the biological signal. The unit of the biological signal axis may be, for example, millivolt (mV), microvolt (μV), beats per minute (bpm), millimeter of mercury (mmHg), Pascal (Pa), degrees Celsius (°C), or other units. The vertical axis may be a time axis, and the horizontal axis may be a biological signal axis. In addition, the signal waveform may be a waveform represented two-dimensionally or a waveform represented three-dimensionally. In the case of a three-dimensional waveform, the signal waveform is, for example, a graph obtained by plotting time-series data with the x-axis as a time axis and the y-axis and the z-axis as biological signal axes. The signal waveform typically includes the axis of the graph, i.e., the reference line of the signal waveform, but may not include the axis of the graph.

The diagnosis support system 1 acquires, for example, a signal waveform of a biological signal measured by a predetermined medical apparatus (not illustrated). Here, the “medical apparatus” refers to an apparatus capable of measuring a signal waveform of a biological signal, and is not strictly limited to an apparatus used for medical care. In addition, the medical apparatus may be an electrocardiograph, an electroencephalograph, an electromyograph, a pulse wave meter, or a thermometer, or may be other apparatuses. The medical apparatus may be a wearable apparatus. A wearable apparatus is a computer apparatus that can be worn by a user and has a built-in sensor for measuring a signal waveform of a predetermined biological signal. The wearable apparatus may be, for example, a smartwatch, a smart glass, a smart ring, a head-mounted display, or may be other wearable apparatuses.

The medical apparatus may output an image including the measured signal waveform. That is, the medical apparatus may generate an image including the measured signal waveform. In the present disclosure, the image is referred to as an “original image”. The format of the original image may be, for example, Joint Photographic Experts Group (JPEG), Portable Network Graphics (PNG), Graphics Interchange Format (GIF), or the like, or may be Portable Document Format (PDF). Furthermore, the original image may have a format conforming to a Medical waveform Format Encoding Rule (MFER). The formats may be the same or may be different between the medical apparatuses. One original image may include one signal waveform or a plurality of signal waveforms.

The original image may include information other than the signal waveform. For example, the original image may include time information at which the signal waveform is acquired, a name of a medical apparatus that measures the signal waveform, a name of a company that manufactures the medical apparatus, and the like. In addition, the original image may include a predetermined reference object for the signal waveform. The reference object is an object drawn such that a predetermined numerical value represented in a predetermined unit has a predetermined size. The reference object may be represented by, for example, a grid line. For the size of one frame in the grid line, for example, the length in the horizontal axis direction represents the length of time, and the length in the vertical axis direction represents the magnitude of the biological signal. The grid lines may be drawn in the background of the signal waveform in the original image. The reference object may also be a calibration wave. The calibration wave is, for example, a rectangular object used for calibration in the case of measuring an electrocardiogram or an electromyogram. In the original image, the calibration wave may be arranged, for example, in such a way as to be adjacent to the signal waveform.

That is, the medical apparatus may not generate an image including the measured signal waveform. For example, the medical apparatus may output the measured signal waveform to a predetermined server apparatus (not illustrated). In other words, the medical apparatus may transmit the measured signal waveform to a predetermined server apparatus. Here, the signal waveform transmitted by the medical apparatus may be in an image format or a numerical format. Then, the server apparatus may generate the original image using the received signal waveform. In this case, the server apparatus can output the original image including the signal waveform. In other words, the server apparatus can transmit the original image including the signal waveform.

The diagnosis support system 1 includes an acquisition unit 11 and an adjustment unit 12. The acquisition unit 11 and the adjustment unit 12 are typically software or modules in which processing is executed by a processor executing a program stored in a memory. The acquisition unit 11 and the adjustment unit 12 may be hardware such as a circuit or a chip. Furthermore, the acquisition unit 11 and the adjustment unit 12 may be configured by different computer apparatuses.

The acquisition unit 11 and the adjustment unit 12 are connected in such a way as to enable data communication. Data communication between the acquisition unit 11 and the adjustment unit 12 may be performed via a wired or wireless network. The data communication may be performed in the same computer apparatus, may be performed via an Internet line, or may be performed by using a short-range wireless communication technique. A type of communication protocol in data communication is not limited.

The acquisition unit 11 acquires an original image including signal waveforms of a plurality of types of biological signals for the same user. The acquisition unit 11 is also referred to as an acquisition means. The acquisition unit 11 may directly acquire the original image from a predetermined medical apparatus that measures the biological signal, or may acquire the original image from a server apparatus or a communication apparatus (not illustrated). For example, in a case where the medical apparatus can generate the original image, the acquisition unit 11 can directly acquire the original image from the medical apparatus. Furthermore, in a case where the server apparatus generates the original image, the acquisition unit 11 can acquire the original image from the server apparatus. Here, the “plurality of types of biological signals” means a plurality of biological signals of different types. The “plurality of types of biological signals” may be, for example, an “electrocardiogram and electroencephalogram”, “pulse, respiration and blood pressure”, or a combination of other biological signals. The “original image including signal waveforms of a plurality of types of biological signals” means an original image including a plurality of signal waveforms.

The original image acquired by the acquisition unit 11 includes a plurality of signal waveforms. In a case where one original image includes one signal waveform, the acquisition unit 11 acquires a plurality of original images. Furthermore, in a case where one original image includes a plurality of signal waveforms, the acquisition unit 11 may acquire one original image or may acquire a plurality of original images. In a case where the acquisition unit 11 acquires a plurality of original images, they may have the same format or may have different formats.

At least one scale of at least two signal waveforms having different types of biological signals among a plurality of signal waveforms included in one or two or more original images acquired by the acquisition unit 11 are different from each other. “Scales are different from each other” means that, for example, scales of related axes for two signal waveforms are different from each other. For example, for two signal waveforms, scales of the time axis may be different from each other, scales of the biological signal axis may be different from each other, or both scales may be different from each other. Specifically, “scales are different” means that, for example, a range of related axis, a length of axis, or a unit of scale is different in two signal waveforms. For example, in a case where the acquisition unit 11 acquires one or two or more original images including three or more signal waveforms, scales of related axes may be different for each of the signal waveforms.

The adjustment unit 12 adjusts at least one of the scales of the plurality of signal waveforms in the original image acquired by the acquisition unit 11 based on a predetermined adjustment criterion. The adjustment unit 12 is also referred to as an adjustment means. The adjustment unit 12 can adjust at least one scale of at least two signal waveforms having different types of biological signals among the plurality of signal waveforms based on a predetermined adjustment criterion. Specifically, for example, the adjustment unit 12 can adjust the range of axis, the length of axis, and the unit of scale for signal waveforms having different scales from each other among a plurality of signal waveforms. As a result, the adjustment unit 12 can, for example, change the shape of the signal waveform. The adjustment criterion can be arbitrarily determined. For example, the adjustment criterion can be determined in advance by the user.

FIG. 2 is a flowchart illustrating a flow of a diagnosis support method by the diagnosis support system 1. First, the acquisition unit 11 acquires an original image including signal waveforms of a plurality of types of biological signals for the same user (S101). Here, the acquisition unit 11 may directly acquire the original image from the medical apparatus, or may acquire the original image from the server apparatus or the communication apparatus. Next, the adjustment unit 12 adjusts at least one of the scales of the plurality of signal waveforms in the original image acquired by the acquisition unit 11 based on a predetermined adjustment criterion (S102).

As described above, in the first example embodiment, at least one of the scales of the signal waveforms included in the original images of the plurality of types of biological signals for the same user can be adjusted based on a predetermined adjustment criterion. Here, at least one scale of at least two signal waveforms having different types of biological signals among the plurality of signal waveforms included in the original image is different from each other. By optimizing by adjusting the scale in such signal waveform, a doctor or a predetermined computer system (not illustrated) can compare a plurality of types of signal waveforms. By comparing a plurality of types of signal waveforms, a doctor or the like can diagnose various diseases. Thus, in the first example embodiment, it is possible to support diagnosis by a doctor or the like in which various biological signals are integrated.

### Second Example Embodiment

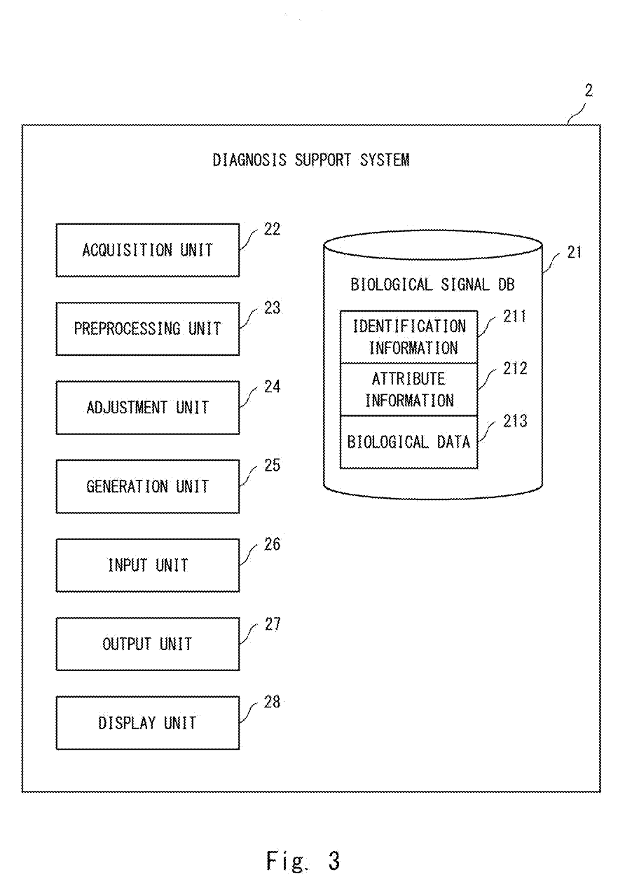

Next, a second example embodiment will be described. The second example embodiment is a specific example of the first example embodiment described above. FIG. 3 is a block diagram illustrating a configuration of the diagnosis support system 2 according to the present disclosure. Similarly to the diagnosis support system 1 according to the first example embodiment, the diagnosis support system 2 is an information processing system for adjusting at least one of scales of signal waveforms included in the original images of a plurality of types of biological signals for the same user based on a predetermined adjustment criterion. The diagnosis support system 2 can generate a diagnostic image including a signal waveform whose scale has been adjusted. Then, the diagnosis support system 2 can output a diagnostic result of medical diagnosis for the user by inputting the image to a learned model learned by a predetermined machine learning algorithm. In addition, the diagnosis support system 2 can display the generated diagnostic image to a doctor or the like.

The diagnosis support system 2 includes a biological signal database (DB) 21, an acquisition unit 22, a preprocessing unit 23, an adjustment unit 24, a generation unit 25, an input unit 26, an output unit 27, and a display unit 28. These elements are typically software or modules in which processing is executed by a processor executing a program stored in a memory. These elements in the diagnosis support system 2 may be hardware such as a circuit or a chip. These elements may be configured by the same computer apparatus or may be configured by different computer apparatuses.

The elements in the diagnosis support system 2 are connected in such a way as to be able to communicate data with each other as necessary. The data communication may be performed via a wired or wireless network. The data communication may be performed in the same computer apparatus, may be performed via an Internet, or may be performed by using a short-range wireless communication technology. A type of communication protocol in data communication is not limited.

The biological signal DB 21 is configured by a storage apparatus such as a hard disk or a flash memory. The biological signal DB 21 may have a function of connecting to a storage apparatus or a storage system such as a cloud storage, a Network Attached Storage (NAS), a Storage Area Network (SAN), or the like via a network. The biological signal DB 21 is a database that stores biological data of the user. Here, the user may or may not refer to a person to be measured of the biological signal to be adjusted by the diagnosis support system 2. That is, the biological signal DB 21 may store biological data of a person unrelated to the person targeted by the diagnosis support system 2. The biological signal DB 21 stores a plurality of pieces of information regarding the biological data in association with each other. Specifically, the biological signal DB 21 stores the identification information 211, the attribute information 212, and the biological data 213 in association with each other.

The identification information 211 is user identification information stored in the biological signal DB 21. The identification information 211 may be, for example, an identification (ID) of the user, a name of the user, or both. The attribute information 212 is information regarding the attribute of the user. The attribute of the user may include, for example, basic attributes such as age, gender, height, weight, and nationality of the user. Furthermore, the attribute of the user may include a medical attribute such as a medical history, a drug administration history, a lifestyle, and information regarding allergy of the user. The attribute of the user is not limited thereto.

The biological data 213 is information regarding the biological data of the user. The “biological data” is data regarding a biological signal of the user. The biological data 213 may include data regarding a biological signal targeted by the diagnosis support system 2. For example, the biological data 213 may be data regarding an electrocardiogram, an electroencephalogram, an electromyogram, a pulse, a blood pressure, respiration, and a body temperature, or may be data regarding other biological signals. The biological data 213 may be signal waveform data of a biological signal, numerical data of a biological signal, or both. For example, the biological data 213 may be signal waveform data similar to the signal waveform to be adjusted by the diagnosis support system 2, or may be data representing time-series data of the biological signal in a table format. Furthermore, the biological data 213 may include normal data for a predetermined biological signal. The “normal biological data” is data regarding a biological signal observed among users in a healthy state. For example, the biological data 213 may include data of healthy persons. Furthermore, the biological data 213 may include data statistically determined based on healthy persons. That is, the biological data 213 may not be data of a specific individual, but may be data statistically determined by data of an unspecified number of users. For example, the normal biological data may be a reference value for a predetermined biological signal.

The acquisition unit 22 acquires an original image including signal waveforms of a plurality of types of biological signals for the same user. The acquisition unit 22 is also referred to as an acquisition means. That is, the acquisition unit 22 corresponds to the acquisition unit 11 in the diagnosis support system 1. Since the original image acquired by the acquisition unit 22 is similar to the original image acquired by the acquisition unit 11 in the diagnosis support system 1, the description thereof will be omitted. Similarly to the acquisition unit 11, the acquisition unit 22 may acquire the original image from a medical apparatus (not illustrated) capable of measuring the signal waveform of the biological signal, or may acquire the original image from a server apparatus or a communication apparatus (both are not illustrated).

The acquisition unit 22 can further acquire the attribute of the user who is the measurement target of the biological signal included in the original image. The attribute of the user is an attribute included in the attribute information 212 stored in the biological signal DB 21. That is, the acquisition unit 22 may acquire the basic attribute of the user, may acquire the medical attribute, or may acquire both of them. The acquisition unit 22 may acquire the attribute of the user by accepting an input of the attribute of the user from the user via, for example, a predetermined input apparatus (not illustrated). The input apparatus may be, for example, a mouse, a keyboard, a touch panel, a microphone, or another input apparatus. The acquisition unit 22 may acquire the attribute of the user from a person other than the user who is the measurement target of the biological signal included in the original image. Furthermore, in a case where the user is a patient of a predetermined medical institution, the acquisition unit 22 may acquire the attribute of the user by communicating with an electronic medical record system (not illustrated) managed by the medical institution. The acquisition unit 22 may also acquire personal information such as the user’s name.

The preprocessing unit 23 performs predetermined preprocessing on the original image acquired by the acquisition unit 22. The preprocessing unit 23 is also referred to as a preprocessing means. For example, it is assumed that the original image acquired by the acquisition unit 22 includes the following contents. That is, it is assumed that the original image includes a plurality of types of signal waveforms in which grid lines are drawn in the background. In addition, it is assumed that calibration waves are arranged adjacent to each of the signal waveforms. Furthermore, it is assumed that the original image includes the time information at which the signal waveform is acquired and the name of the medical apparatus that measured the signal waveform.

First, the preprocessing unit 23 extracts a portion where a signal waveform is drawn from the original image. For example, in a case where the region of the signal waveform and the other regions are defined in advance in the original image, the preprocessing unit 23 extracts the region of the signal waveform from the original image. Furthermore, the preprocessing unit 23 may specify the region of the signal waveform by analyzing the original image. Then, the preprocessing unit 23 may extract a region of the signal waveform from the original image. Furthermore, the preprocessing unit 23 may cause the user to designate the extraction range. In this case, the preprocessing unit 23 may accept an input of the extraction range from the user via a predetermined input apparatus. Then, the preprocessing unit 23 can extract the range designated by the user as the region of the signal waveform. The preprocessing unit 23 may include a calibration wave in the region of the signal waveform to be extracted.

Next, the preprocessing unit 23 can remove the grid lines drawn in the background of the extracted signal waveform. For example, the preprocessing unit 23 can remove the grid lines in the background by setting an appropriate binarization threshold value for the extracted signal waveform and performing binarization processing. Furthermore, in a case where the extracted signal waveform includes a calibration wave and other symbols, the preprocessing unit 23 can remove these elements. For example, the preprocessing unit 23 may remove elements other than the signal waveform by performing mask processing on a region where these elements are displayed. Here, in a case where how the elements to be removed are arranged with respect to the signal waveform is defined in advance, the preprocessing unit 23 can remove a region other than the signal waveform by performing mask processing on the region defined in advance. Even in a case where the arrangement of elements to be removed is not determined in advance, the preprocessing unit 23 may remove detected characters and the like by, for example, detecting characters and the like other than the signal waveform by Optical Character Recognition (OCR) or the like. In addition, the preprocessing unit 23 may detect an element other than the signal waveform by performing pattern matching or the like. As the mask processing, the preprocessing unit 23 may perform processing of setting the target region to black, may perform processing of setting the target region to white, or may perform processing of setting the target region to other colors. Furthermore, instead of or in addition to the mask processing, the preprocessing unit 23 may perform processing of narrowing down the region in such a way that elements other than the signal waveform are not displayed. In other words, the preprocessing unit 23 may further extract the region of the signal waveform in such a way that elements other than the signal waveform are not displayed. Image data including the signal waveform extracted in this manner is referred to as “extracted image data”.

Next, the preprocessing unit 23 detects a pixel group related to a signal waveform in the extracted image data. For example, the preprocessing unit 23 can detect a pixel group by allocating a predetermined coordinate value to each pixel in the extracted image data. Specifically, the preprocessing unit 23 can allocate a coordinate value to each pixel of the extracted image data, assuming that the coordinates of the pixel at the reference position such as the upper left end of the extracted image data are (0, 0), the rightward direction of the extracted image data is the positive direction of the x-axis, the downward direction is the positive direction of the y-axis, and the interval between adjacent pixels is 1. Then, the preprocessing unit 23 can detect a pixel group including adjacent pixels as a pixel group related to the signal waveform.

In a case where characters and the like exist in the extracted image data, the preprocessing unit 23 may detect, as a pixel group related to the signal waveform, a pixel group satisfying a predetermined condition. For example, the preprocessing unit 23 may set, as a pixel group related to the signal waveform, a pixel group in which the number of elements is equal to or greater than a threshold value. Furthermore, the preprocessing unit 23 may set, as a pixel group related to the signal waveform, a pixel group in which the distance of the pixels from the left end to the right end is equal to or greater than a threshold value.

Furthermore, in a case where the extracted image data includes a calibration wave or an axis of a graph, the preprocessing unit 23 may detect a pixel group related to the signal waveform by detecting these elements and the signal waveform in a distinguished manner. For example, the preprocessing unit 23 may detect a pixel group related to the signal waveform by performing pattern matching or the like and distinguishing the calibration wave and the axis of a graph from a signal waveform.

The preprocessing unit 23 can derive a reference line in the signal waveform. The “reference line” is a line serving as a reference of time and a value of a biological signal with respect to a signal waveform. Specifically, the reference line refers to a line at which the time and the value of the biological signal become zero. Here, the “line at which the time becomes zero” may mean a line at which the time or date becomes a predetermined reference value. That is, in a case where the x-axis (horizontal axis) of the signal waveform is the time axis and the y-axis (vertical axis) is the biological signal axis, the reference line at which the value of the biological signal becomes zero is a line extending in the x-axis direction, and the reference line at which the time becomes zero is a line extending in the y-axis direction. In a case where the signal waveform includes the x-axis and the y-axis in the original image, the reference line indicates these axes. In a case where the signal waveform in the original image includes the x-axis and the y-axis, the preprocessing unit 23 derives these axes as reference lines. In a case where the signal waveform in the original image does not include the x-axis and the y-axis, the preprocessing unit 23 may derive the reference line based on, for example, coordinates of a pixel at the left end in the signal waveform. That is, the preprocessing unit 23 can derive two reference lines at which the value of the biological signal and the time become zero based on the pixel at the left end in the signal waveform.

The preprocessing unit 23 can specify the scale of the detected signal waveform. For example, a case where an x-axis (horizontal axis) of a signal waveform is a time axis and a y-axis (vertical axis) is a biological signal axis is considered. The preprocessing unit 23 can specify the scale of the signal waveform by using, for example, a grid line or a calibration wave included in the original image as a reference object. For example, in a case where the correspondence relationship between the size in the y-axis direction in one frame of the grid line and the value of the biological signal in the signal waveform is known, the preprocessing unit 23 can specify the value of the biological signal per pixel. Similarly, in a case where the correspondence relationship between the size in the x-axis direction in one frame of the grid line and the length of time is known, the preprocessing unit 23 can specify the length of the elapsed time per pixel. Similarly, the preprocessing unit 23 may use the calibration wave as the reference object. In a case where the value of the biological signal with respect to the height of the predetermined calibration wave is known, the preprocessing unit 23 can specify the value of the biological signal per pixel by using the calibration wave. In this manner, the preprocessing unit 23 can specify the values in the x-axis direction and the y-axis direction in the signal waveform based on the distance from the reference line.

The preprocessing unit 23 may generate numerical data based on the values in the x-axis direction and the y-axis direction specified for the signal waveform. Specifically, the preprocessing unit 23 can calculate the numerical data of each pixel by multiplying the distance between the reference line and the x-axis direction and the y-axis direction of each pixel by the specified scale. The preprocessing unit 23 can generate the value in the x-axis direction and the value in the y-axis direction obtained in this manner, for example, as tabular data.

The adjustment unit 24 adjusts at least one of the scales of the plurality of signal waveforms in the original image acquired by the acquisition unit 22 based on a predetermined adjustment criterion. The adjustment unit 24 is also referred to as an adjustment means. That is, the adjustment unit 24 corresponds to the adjustment unit 12 in the diagnosis support system 1. The adjustment unit 24 can adjust the scale of the signal waveform based on the values in each of the axial directions in the signal waveform specified by the preprocessing unit 23. The adjustment of the scale means, for example, generating a signal waveform on a new scale by substantially expanding and contracting the signal waveform in the original image. In addition, the adjustment of the scale may mean adding, removing, or modifying a scale on an axis of a graph.

The adjustment unit 24 may adjust at least one of scales of some signal waveforms or may adjust at least one of scales of all signal waveforms among the signal waveforms of the plurality of types of biological signals acquired by the acquisition unit 22. Furthermore, the adjustment unit 24 may adjust some scales of the signal waveform to be adjusted, or may adjust all scales. That is, the adjustment unit 24 can adjust an arbitrary scale in an arbitrary signal waveform among the scale of the time axis and the scale of the biological signal axis in the signal waveforms of the plurality of types of biological signals. For example, the adjustment unit 24 may adjust only the scale of the time axis or may adjust only the scale of the biological signal axis. The adjustment unit 24 can adjust at least one of the scale of the time axis in the signal waveform and the scale of the biological signal axis independently of the other scale. That is, the adjustment degree of the scale of the time axis and the adjustment degree of the scale of the biological signal axis may be independent of each other. Here, the “adjustment degree” is the degree of change between the scale of each axis before adjustment and the scale of each axis after adjustment. The adjustment degree is, for example, a degree of change in length in each axial direction in the signal waveform. The adjustment unit 24 may adjust the adjustment degree of the scale of the time axis to be larger than the adjustment degree of the scale of the biological signal axis. Furthermore, the adjustment unit 24 may adjust the adjustment degree of the scale of the biological signal axis to be larger than the adjustment degree of the scale of the time axis.

The adjustment unit 24 can adjust the scale of the time axis. For example, the adjustment unit 24 can unify the scales of time axes in signal waveforms of a plurality of types of biological signals. In other words, the adjustment unit 24 can unify the lengths in the time axis direction in the plurality of signal waveforms. For example, the adjustment unit 24 can adjust the scale on the time axis by enlarging one signal waveform in the time axis direction. For example, assume a case where the acquisition unit 22 acquires the signal waveform of the blood pressure and the signal waveform of the electrocardiogram, the x-axis of each signal waveform representing the time axis and the y-axis representing the biological signal axis. In a case where the signal waveform of the blood pressure indicates a waveform for 5 seconds and the signal waveform of the electrocardiogram indicates a waveform for 3 seconds, and the lengths in the x-axis direction of the two waveforms are the same due to a difference in the number of seconds per pixel, the adjustment unit 24 can, for example, adjust the signal waveform of the electrocardiogram. Specifically, the adjustment unit 24 can unify the number of seconds per pixel in the signal waveform of the electrocardiogram to the number of seconds per pixel in the signal waveform of the blood pressure by multiplying the number of seconds per pixel by 5/3 for the signal waveform of the electrocardiogram. As a result, the adjustment unit 24 can adjust the scale of the time axis in the signal waveforms of the two types of biological signals. In the above example, the adjustment unit 24 may adjust the signal waveform of the blood pressure or may adjust the signal waveforms of both the electrocardiogram and the blood pressure. The unit of the time axis may not be seconds. The unit of the time axis may be minutes, hour (Hour), day, or millisecond.

The adjustment unit 24 may adjust the scale such that the plurality of signal waveforms can be compared without unifying the scales of the time axis in the signal waveform. For example, the adjustment unit 24 may adjust two signal waveforms in such a way that the length in one time axis direction is an integral multiple of the length in the other time axis direction. Furthermore, the adjustment unit 24 may adjust one signal waveform independently of the other signal waveform. For example, the adjustment unit 24 may adjust the length in the time axis direction of the signal waveform to be twice the length of the original waveform. The adjustment unit 24 can adjust the length in the time axis direction of the signal waveform to an arbitrary length by changing the elapsed time per pixel.

Furthermore, the adjustment unit 24 can adjust the scale of the signal waveform based on the normal biological data 213 for the target biological signal by referring to the biological signal DB 21. Specifically, the adjustment unit 24 can adjust the scale of the time axis in the signal waveform based on the normal biological data 213 for the target biological signal. That is, the adjustment unit 24 can adjust the scale of the time axis in the signal waveform by extracting the normal biological data 213 from the biological signal DB 21. Here, the normal biological data 213 may be data related to the same user as the user who is the measurement target of the biological signal included in the original image, may be data related to a different user, or may be data partially including the data of the user who is the measurement target of the biological signal included in the original image.

Here, the adjustment unit 24 can adjust the scale of the time axis in the signal waveform in consideration of the attribute of the user acquired by the acquisition unit 22. That is, the adjustment unit 24 can adjust the scale of the time axis based on the normal biological data 213 in the attribute of the user acquired by the acquisition unit 22. In this case, for example, the adjustment unit 24 searches whether the biological data 213 having the attribute that matches the attribute of the user acquired by the acquisition unit 22 is stored in the biological signal DB 21 using the attribute information 212 in the biological signal DB 21. In a case where the acquisition unit 22 has acquired a plurality of attributes for the user, the adjustment unit 24 may search whether the biological data 213 of the user having the attribute information 212 with which all the attributes match is stored in the biological signal DB 21. Furthermore, the adjustment unit 24 may search whether the biological data 213 of the user having the attribute information 212 with which some attributes match is stored in the biological signal DB 21. Furthermore, the adjustment unit 24 may search the biological data 213 of the user having the attribute information 212 associated with the attribute of the user acquired by the acquisition unit 22. The “associated attribute” means that, for example, one attribute is within a predetermined range with respect to the other attribute. For example, in a case where the age of the user acquired as the attribute by the acquisition unit 22 is 40 years old, the adjustment unit 24 may search for users up to 5 year-old difference from the biological signal DB 21. As a result of the search, the normal biological data 213 having the attribute information 212 that matches or is associated with the attribute of the user acquired by the acquisition unit 22 is extracted from the biological signal DB 21, whereby the adjustment unit 24 can adjust the scale of the time axis in the signal waveform.

For example, it is assumed that the length of the signal waveform in the time axis direction for a certain biological signal is adjusted with the length for five cycles in a normal biological signal as a reference. In this case, the adjustment unit 24 grasps the length for five cycles in the normal biological signal by extracting the normal biological data 213 from the biological signal DB 21. Then, the adjustment unit 24 adjusts the elapsed time per pixel in such a way that the length in the normal biological signal becomes the length of the time axis in the signal waveform to be adjusted. As a result, the adjustment unit 24 can adjust the scale of the time axis in the signal waveform based on the normal biological data 213.

The adjustment unit 24 can adjust not only the time axis but also the scale of the biological signal axis. For example, the adjustment unit 24 can unify scales of biological signal axes in the signal waveforms of a plurality of types of biological signals. In other words, the adjustment unit 24 can unify the lengths from the minimum value to the maximum value of the biological signal in the plurality of signal waveforms. In other words, the adjustment unit 24 can unify the widths from the minimum value to the maximum value of the biological signal in the plurality of signal waveforms. For example, the adjustment unit 24 can adjust the scale of the biological signal axis by enlarging one signal waveform in the biological signal axis direction. Assume a case where the plurality of signal waveforms are the signal waveform of the blood pressure and the signal waveform of the electrocardiogram. In a case where the length in the biological signal axis direction from the minimum value to the maximum value of the blood pressure in the signal waveform of the blood pressure is 2 cm, and the length in the biological signal axis direction from the minimum value to the maximum value of the potential due to the myocardium in the signal waveform of the electrocardiogram is 1 cm, for example, the adjustment unit 24 can adjust the signal waveform of the electrocardiogram. Specifically, the adjustment unit 24 can unify the lengths from the minimum value to the maximum value of the potential in the electrocardiogram and the lengths from the minimum value to the maximum value of the blood pressure by setting the potential per pixel of the signal waveform of the electrocardiogram to 1/2 times. As a result, the adjustment unit 24 can adjust the scale of the biological signal axis in the signal waveforms of the two types of biological signals. In the above example, the adjustment unit 24 may adjust the signal waveform of the blood pressure or may adjust the signal waveforms of both the electrocardiogram and the blood pressure. Furthermore, in a case where the signal waveform includes the axis of the graph, the adjustment unit 24 may perform adjustment by unifying the lengths up to the maximum value of the biological signal from the time axis.

The adjustment unit 24 may not unify the scale of the biological signal axis in the signal waveform. For example, the adjustment unit 24 may adjust the two signal waveforms in such a way that the length in one biological signal axis direction is an integral multiple of the length in the other biological signal axis direction. Furthermore, for example, the adjustment unit 24 may adjust the length in the biological signal axis direction of the signal waveform to be twice the length of the original waveform. The adjustment unit 24 can adjust the length in the biological signal axis direction of the signal waveform to an arbitrary length by changing the value of the biological signal per pixel.

The adjustment unit 24 can adjust the scale of the biological signal axis in the signal waveform based on the normal biological data 213 for the target biological signal by referring to the biological signal DB 21. The adjustment unit 24 can adjust the scale of the biological signal axis in the signal waveform based on, for example, the maximum value of the values of the biological signals in the normal biological data 213. In this case, the adjustment unit 24 compares the maximum value of the biological signal in the normal biological data 213 with the maximum value in the biological signal axis direction in the signal waveform. Assume a case where the maximum value in the normal biological data 213 is smaller than the maximum value in the biological signal axis direction in the signal waveform. In this case, for example, the adjustment unit 24 can adjust the length in the biological signal axis direction in the signal waveform by changing the value of the biological signal per pixel such that the maximum value in the normal biological data 213 is located substantially at the center of the biological signal axis in the signal waveform. In addition, assume a case where the maximum value in the normal biological data 213 is greater than the maximum value in the biological signal axis direction in the signal waveform. In this case, for example, the adjustment unit 24 can adjust the length in the biological signal axis direction in the signal waveform by changing the value of the biological signal per pixel such that the maximum value in the normal biological data 213 becomes the length of the biological signal axis in the signal waveform.

Furthermore, similarly to the scale of the time axis, the adjustment unit 24 can adjust the scale of the biological signal axis in the signal waveform in consideration of the attribute of the user acquired by the acquisition unit 22. That is, the adjustment unit 24 can adjust the scale of the biological signal axis based on the normal biological data 213 in the attribute of the user acquired by the acquisition unit 22. The adjustment unit 24 searches whether the biological data 213 of the user having the attribute information 212 that matches or is associated with the attribute of the user acquired by the acquisition unit 22 is stored in the biological signal DB 21. As a result of the search, the adjustment unit 24 can adjust the scale of the biological signal axis in the signal waveform by extracting the normal biological data 213 having the target attribute from the biological signal DB 21.

In a case where a plurality of types of biological signals included in the original image have a plurality of units, the adjustment unit 24 can adjust the scale of the biological signal axis by converting the plurality of units into predetermined common units for the scale of the biological signal axis in the target signal waveform. The “case where the plurality of types of biological signals have a plurality of units” is a case where the units of at least two biological signals of the plurality of types of biological signals have different units. Here, the “unit” includes, for example, a basic unit, a derived unit, and other non-SI units in the international unit system. In a case where different prefixes (SI prefixes) are used even if the basic units are the same, they may be regarded as different units. For example, mV and μV may be different units. Furthermore, the “common unit” includes a dimensionless number.

For example, the adjustment unit 24 may adjust the scale of the biological signal axis in the signal waveform by converting the signal waveform of the blood pressure represented by mmHg and the signal waveform of the electrocardiogram represented by mV into percentages (%) with respect to a predetermined normal value. For example, the adjustment unit 24 can adjust the scale with the maximum value of the blood pressure and the potential of the myocardium in the normal biological data 213 stored in the biological signal DB 21 as 100%. In this case, for example, assuming that the length of the biological signal axis in the signal waveforms of the blood pressure and the electrocardiogram is 100%, the adjustment unit 24 calculates the ratio of the blood pressure of the signal waveform and the potential of the myocardium with respect to the maximum values of the blood pressure and the potential of the myocardium in the normal biological data 213. As a result, the adjustment unit 24 can adjust the scale by converting the signal waveforms of the two types of biological signals into the percentage that is the common unit. Here, the adjustment unit 24 may perform scale adjustment with the maximum value of the values of the biological signal in the normal biological data 213 as 1. That is, the adjustment unit 24 may normalize the scale of the biological signal axis based on the normal biological data 213 for the signal waveforms of the plurality of types of biological signals.

In a case where the target signal waveform includes a graph axis such as a time axis or a biological signal axis, the adjustment unit 24 can add or delete a scale on the axis of the graph. Similarly, the adjustment unit 24 can change the position of the scale on the axis of the graph. For example, the adjustment unit 24 can add, delete, or change the position of the scale such that the scales of the time axis or the biological signal axis of the plurality of signal waveforms are the same. Furthermore, in a case where the target signal waveform does not include the axis of the graph, the adjustment unit 24 can add the axis of the graph. Furthermore, in a case where the target signal waveform includes an axis of the graph, the adjustment unit 24 can delete the axis.

The adjustment unit 24 can generate a signal waveform in which the scale of each axis is adjusted by directly expanding and contracting the signal waveform in the original image based on the value in each axial direction in the signal waveform specified by the preprocessing unit 23. Furthermore, the adjustment unit 24 can newly generate a signal waveform in which the scale of each axis is adjusted based on numerical data for the signal waveform in the original image generated by the preprocessing unit 23. The signal waveform generated by the adjustment unit 24 adjusting the scale is typically an image. The signal waveform generated by the adjustment unit 24 may have the same format as that of the original image or may have a different format.

The adjustment criterion of the scale of the time axis and the scale of the biological signal axis in the signal waveform by the adjustment unit 24 can be arbitrarily determined. For example, the adjustment criterion may be determined by a medical worker such as a doctor, or may be determined by a predetermined computer system (not illustrated). The adjustment unit 24 transmits the scale-adjusted signal waveform to the generation unit 25.

The generation unit 25 generates a diagnostic image including a signal waveform whose scale has been adjusted by the adjustment unit 24. The generation unit 25 is also referred to as a generation means. The “diagnostic image” is an image used in a case where some kind of medical diagnosis is made for a user who is a measurement target of the biological signal included in the original image The subject performing the medical diagnosis may be a doctor or a learned model to be described later. That is, the generation unit 25 can generate a diagnostic image for medical diagnosis by a doctor or a learned model. The format of the diagnostic image may be, for example, JPEG, PNG, GIF, or PDF. In addition, the format of the diagnostic image may conform to MFER.

For example, the generation unit 25 may generate the signal waveform received from the adjustment unit 24 as it is as the diagnostic image. That is, the generation unit 25 may generate a plurality of diagnostic images with each of the plurality of signal waveforms newly generated by the adjustment unit 24 as a diagnostic image. In addition, the generation unit 25 may generate one diagnostic image by arranging a plurality of signal waveforms generated by the adjustment unit 24 in one image. The generation unit 25 can generate one diagnostic image by, for example, arranging a plurality of signal waveforms at equal intervals in one image.

The generation unit 25 can generate a diagnostic image based on a layout defined in advance for the arrangement of the signal waveforms received from the adjustment unit 24. For example, the generation unit 25 may arrange a plurality of signal waveforms received from the adjustment unit 24 at a position defined in advance in the image. Then, the generation unit 25 may generate the diagnostic image by adding personal information such as the name of the measurement target user related to the signal waveform to a predetermined position.

In addition, the generation unit 25 can generate a diagnostic image by superimposing a plurality of signal waveforms generated by the adjustment unit 24. In other words, the generation unit 25 can generate a diagnostic image by superimposing signal waveforms of a plurality of types of biological signals whose scales have been adjusted. The plurality of signal waveforms adjusted by the adjustment unit 24 have, for example, the length in the time axis direction and the length in the biological signal axis direction unified. In addition, the unit of the biological signal related to these signal waveforms is converted into a predetermined common unit. As a result, the generation unit 25 can generate a diagnostic image in which a plurality of signal waveforms can be easily compared by superimposing these signal waveforms. In a case where the axis of the graph is included in each of the plurality of signal waveforms, the generation unit 25 may generate the diagnostic image by, for example, superimposing the origin in each signal waveform. Even in a case where the signal waveform does not include the axis of the graph, the generation unit 25 may generate the diagnostic image by superimposing arbitrary points in the signal waveform. The generation unit 25 does not need to superimpose the time axis or the biological signal axis in the signal waveform in such a way as to align them. For example, the generation unit 25 may generate the diagnostic image by intentionally shifting and superimposing the time axes of the signal waveforms.

The input unit 26 inputs the diagnostic image to a predetermined learned model. The input unit 26 is also referred to as an input means. The diagnostic image is typically an image generated by the generation unit 25, but is not limited thereto. The diagnostic image may be created by, for example, a medical worker such as a doctor using a signal waveform generated by the adjustment unit 24. In a case where a diagnostic image including signal waveforms of a plurality of types of biological signals is input for the same user, the learned model outputs a diagnostic result of medical diagnosis for the user. The “diagnostic result” can include, for example, good/bad of the health condition of the user, the presence or absence of a suspected disease, the degree of emergency with respect to treatment, a specific disease name, and a diagnosis comment with respect to a diagnostic image. In addition, the diagnostic result may include ground information indicating grounds for good/bad health condition or the like. The diagnostic result may be data in a text format, data in an image format, or data including both of them. The output unit 27 outputs a diagnostic result for the diagnostic image input by the input unit 26 from the learned model. The output unit 27 is also referred to as an output means.

The learning model is learned by a predetermined machine learning algorithm. The learning model is generated based on, for example, a neural network such as a Convolutional Neural Network (CNN). In this case, the learning model includes an input layer, an output layer, and an intermediate layer. A diagnostic image including signal waveforms of a plurality of types of biological signals for the same user is input to the input layer. The output layer outputs a diagnostic result of the medical diagnosis for the input diagnostic image. Parameters of the intermediate layer are learned by using teacher data in which a diagnostic image is the input and a diagnostic result is the output. The learned model generated in this manner is a model for causing a computer to function to input a diagnostic image to the input layer, perform calculation in the intermediate layer, and output a diagnostic result from the output layer. The learned model may be provided in a server apparatus in the diagnosis support system 2 or may be provided in a server apparatus different from the diagnosis support system 2. The input unit 26 and the output unit 27 are data communicably connected to the learned model.

The display unit 28 displays the diagnostic image generated by the generation unit 25 or the diagnostic result output from the learned model by the output unit 27 on the display apparatus. The display unit 28 may display both the diagnostic image generated by the generation unit 25 and the diagnostic result output from the learned model by the output unit 27. The display unit 28 is also referred to as a display means. That is, the display unit 28 outputs display information for displaying the diagnostic image and the diagnostic result on the display apparatus. The display unit 28 may be provided in the same server apparatus as the generation unit 25 or the output unit 27, or may be provided in the display apparatus. The display apparatus is configured by, for example, a liquid crystal panel or organic electroluminescence.

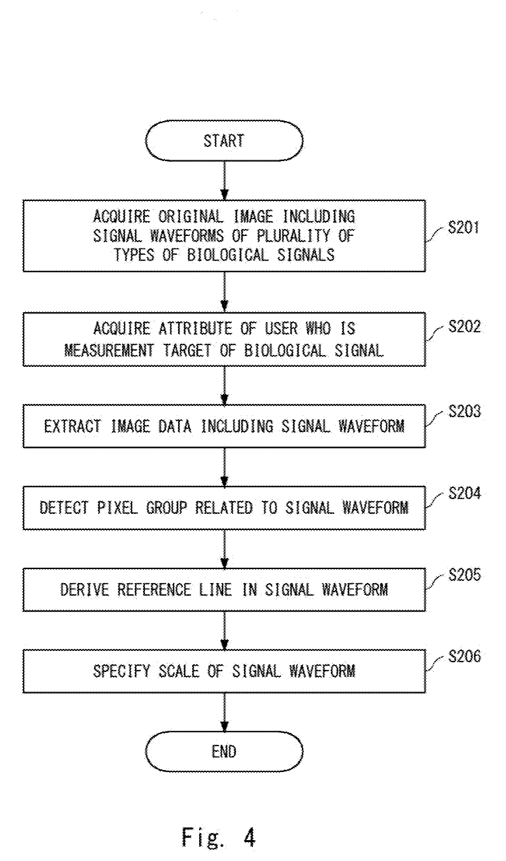

FIG. 4 is a flowchart illustrating an example of a flow of a preprocessing method for an original image. First, the acquisition unit 22 acquires an original image including signal waveforms of a plurality of types of biological signals for the same user (S201). Next, the attribute of the user who is the measurement target of the biological signal included in the original image is acquired (S202). Here, step S202 may be performed before S201, or S201 and S202 may be performed simultaneously. Furthermore, the acquisition unit 22 may acquire personal information such as the name of the user other than the attribute of the user. Thereafter, the preprocessing unit 23 extracts image data including a portion where a signal waveform is drawn from the original image acquired by the acquisition unit 22 (S203). Here, the preprocessing unit 23 can remove grid lines, calibration waves, and other symbols drawn on the background of the signal waveform. Thereafter, the preprocessing unit 23 detects a pixel group related to a signal waveform in the extracted image data (S204). The preprocessing unit 23 can allocate a coordinate value to a pixel group for a signal waveform. Thereafter, the preprocessing unit 23 derives a reference line in the signal waveform (S205). Then, the preprocessing unit 23 specifies the scale of the signal waveform (S206). As a result, the preprocessing unit 23 can specify a value in each axial direction in the signal waveform.

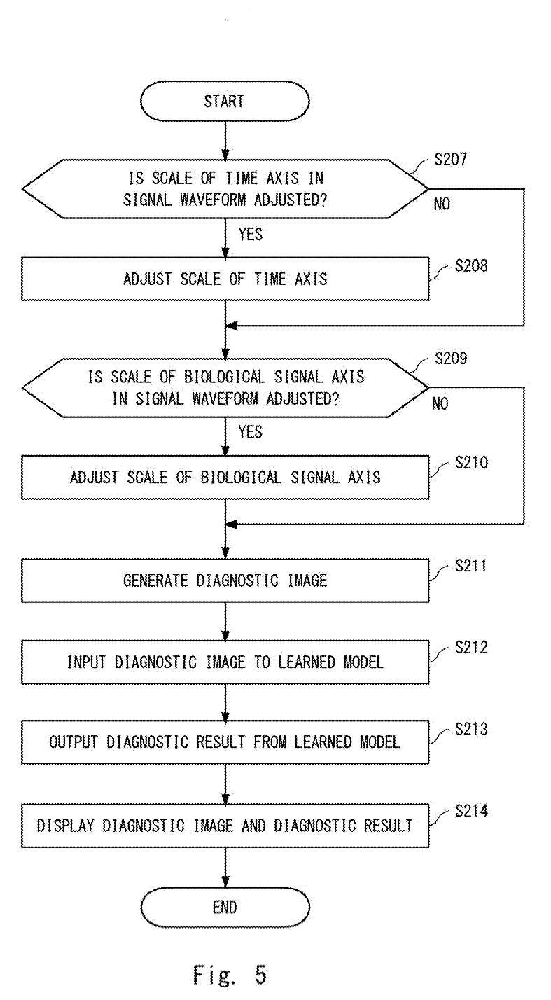

FIG. 5 is a flowchart illustrating an example of a flow of a method of adjusting a scale of a signal waveform. First, the adjustment unit 24 determines whether to adjust the scale of the time axis in the signal waveform (S207). If determined as NO in step S207, the processing proceeds to step S209. On the other hand, if determined as YES in step S207, the adjustment unit 24 adjusts the scale of the time axis by a predetermined adjustment criterion based on the value in the time axis direction in the signal waveform specified by the preprocessing unit 23 (S208). Here, the adjustment unit 24 may adjust the scale of the time axis based on the normal biological data 213 for the biological signal stored in the biological signal DB 21. In particular, the adjustment unit 24 can adjust the scale of the time axis based on the normal biological data 213 in the attribute of the user who is the measurement target of the biological signal. Next, the adjustment unit 24 determines whether to adjust the scale of the biological signal axis in the signal waveform (S209). If determined as NO in step S209, the processing proceeds to step S211. On the other hand, if determined as YES in step S209, the adjustment unit 24 adjusts the scale of the biological signal axis according to a predetermined adjustment criterion based on the value in the biological signal axis direction in the signal waveform specified by the preprocessing unit 23 (S210). Here, similar to the case of the time axis, the adjustment unit 24 may adjust the scale of the biological signal axis based on the normal biological data 213 for the biological signal stored in the biological signal DB 21. In particular, the adjustment unit 24 can adjust the scale of the biological signal axis based on the normal biological data 213 in the attribute of the user. Steps S209 and S210 may be performed before steps S207 and S208, or steps S209 and S110 and steps S207 and S208 may be simultaneously performed.

Thereafter, the generation unit 25 generates a diagnostic image including a signal waveform whose scale has been adjusted by the adjustment unit 24 (S211). For example, the generation unit 25 generates a diagnostic image by superimposing a plurality of signal waveforms. Thereafter, the input unit 26 inputs the diagnostic image generated by the generation unit 25 to the learned model (S212). The input unit 26 may input a diagnostic image created by a medical worker such as a doctor to the learned model. Thereafter, the output unit 27 outputs the diagnostic result for the diagnostic image input by the input unit 26 from the learned model (S213). Thereafter, the display unit 28 causes a predetermined display apparatus to display the diagnostic image generated by the generation unit 25 and the diagnostic result output from the learned model by the output unit 27 (S214). Here, the display unit 28 may cause the display apparatus to display either the diagnostic image generated by the generation unit 25 or the diagnostic result output by the output unit 27.

As described above, according to the second example embodiment, the first example embodiment can be specifically implemented. In recent years, wearable apparatuses such as a smart watch have been widely used. Therefore, an environment in which signal waveforms of various biological signals can be collected without using a dedicated medical apparatus is being prepared. Therefore, a technique for effectively utilizing such a signal waveform is required. On the other hand, in a case where manufacturers of the wearable apparatus and the dedicated medical apparatus are different, scales of acquired signal waveforms may be different from each other. In such a case, there is a possibility that the acquired signal waveforms of the plurality of types of biological signals cannot be effectively utilized. That is, since the scales of the signal waveforms are different from each other, there is a possibility that an appropriate medical diagnosis cannot be performed from the plurality of signal waveforms. According to the second example embodiment, since the diagnosis support system 2 can adjust the scale of the signal waveform, a doctor or a learned model performing a medical diagnosis can compare a plurality of types of signal waveforms. Therefore, a doctor or the like can perform a diagnosis in which various biological signals are integrated by scale adjustment. Therefore, the diagnosis support system 2 can effectively utilize signal waveforms of a plurality of types of biological signals acquired by a wearable apparatus or the like.

In addition, the diagnosis support system 2 can independently adjust the scale of the time axis and the scale of the biological signal axis in the signal waveforms of the plurality of types of biological signals. For example, the diagnosis support system 2 can adjust the scale of the biological signal axis by enlarging the plurality of signal waveforms in the biological signal axis direction. Similarly, the diagnosis support system 2 can adjust the scale of the time axis by enlarging the plurality of signal waveforms in the time axis direction. Furthermore, the diagnosis support system 2 may adjust each scale in the signal waveform such that the adjustment degree of the scale of the time axis is larger than the adjustment degree of the scale of the biological signal axis. Similarly, the diagnosis support system 2 may adjust each scale in the signal waveform such that the adjustment degree of the scale of the biological signal axis is larger than the adjustment degree of the scale of the time axis. Further, the diagnosis support system 2 may adjust only the scale of the time axis or may adjust only the scale of the biological signal axis.

For example, in a case where preprocessing for changing the length and size of the image is performed on the original image to input the image to the learned model learned by a predetermined machine learning algorithm, the preprocessing may be performed such that the ratio of the target image is not different from that of the original image. For example, the preprocessing may be performed in such a way that the aspect ratio of the original image does not change. That is, the preprocessing may be performed such that either one of the length or size of the image depends on the other one of the length or size. In the diagnosis support system 2, the scale of the time axis in the signal waveform and the scale of the biological signal axis can be adjusted independently. As a result, the diagnosis support system 2 can perform free scale adjustment that does not depend on either one of the length or the size for the signal waveform included in the original image. In addition, the diagnosis support system 2 can cause the learned model to perform a highly accurate medical diagnosis by inputting a diagnostic image generated by such an adjustment method to a learned model and outputting a diagnostic result for the diagnostic image from the learned model.

In addition, the diagnosis support system 2 can adjust the scale in the signal waveform based on the normal biological data for the biological signal. As a result, the doctor or the learned model can compare a plurality of signal waveforms based on the normal biological data, and thus, can perform a more accurate medical diagnosis. In particular, the diagnosis support system 2 can adjust the scale based on the normal biological data in the attribute same as or associated with the attribute of the user who is the measurement target of the biological signal included in the original image. As a result, a doctor or the like can perform a diagnosis with higher accuracy.

Furthermore, the diagnosis support system 2 can generate a diagnostic image including a signal waveform whose scale has been adjusted. In addition, the diagnosis support system 2 can generate a diagnostic image by superimposing a plurality of signal waveforms. As a result, the doctor or the like can more easily compare the signal waveforms of the plurality of types of biological signals. In addition, in a case where the learned model performs a diagnosis, the data amount of the diagnostic image can be reduced by superimposing a plurality of signal waveforms, so that the processing cost of the learned model can be reduced.

### Hardware Configuration Example

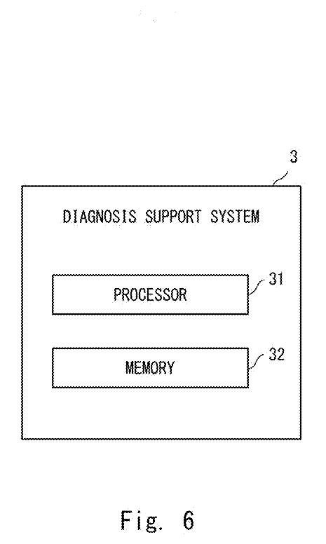

FIG. 6 is a diagram illustrating a hardware configuration example of a diagnosis support system 3. In FIG. 6, the diagnosis support system 3 includes a processor 31 and a memory 32. The processor 31 may be, for example, a microprocessor, a Micro Processing Unit (MPU), or a Central Processing Unit (CPU). The processor 31 may include a plurality of processors. The memory 32 is configured by a combination of a volatile memory and a nonvolatile memory. The memory 32 may include a storage located away from the processor 31. In this case, the processor 31 may access the memory 32 via an Input/Output (I/O) interface (not illustrated).

In the above-described example, a program can be stored by use of various types of non-transitory computer readable medium and provided to a computer. The non-transitory computer readable media include various types of tangible storage medium. Examples of the non-transitory computer readable media include a magnetic storage medium (e.g., magneto-optical disk), a CD-ROM, a CD-R, a CD-R/W, and a semiconductor memory (for example, mask ROM, Programmable ROM (PROM), Erasable PROM (EPROM), flash ROM, and RAM). The program may be provided to the computer by various types of transitory computer readable medium. Examples of the transitory computer readable media include electrical signals, optical signals, and electromagnetic waves. The transitory computer readable media can provide the program to the computer via a wired communication line such as an electric wire and an optical fiber or a wireless communication line. The computer includes various information processing apparatuses such as a PC, a server, a CPU, an MPU, a Field Programmable Gate Array (FPGA), and an Application Specific Integrated Circuit (ASIC).

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

Some or all of the above-described example embodiments may also be described as the following Supplementary Notes, but are not limited to the following Supplementary Notes.

### Supplementary Note 1

A diagnosis support system that supports a medical diagnosis for a user, the diagnosis support system including
an acquisition unit for acquiring an original image including signal waveforms of a plurality of types of biological signals for the same user, and
an adjustment unit for adjusting at least one of scales of the plurality of signal waveforms in the original image based on a predetermined adjustment criterion.

### Supplementary Note 2

The diagnosis support system according to supplementary note 1, in which the adjustment unit adjusts at least one of a scale of a time axis and a scale of a biological signal axis in the signal waveform independently of the other scale.

### Supplementary Note 3

The diagnosis support system according to supplementary note 1 or 2, in which the adjustment unit adjusts the scale based on normal biological data for the biological signal.

### Supplementary Note 4

The diagnosis support system according to supplementary note 3, in which the acquisition unit further acquires an attribute of the user, and
the adjustment unit adjusts the scale based on the normal biological data in the attribute.

### Supplementary Note 5

The diagnosis support system according to any one of supplementary notes 1 to 4, in which
the plurality of types of biological signals include a plurality of units, and
the adjustment unit adjusts a scale of a biological signal axis in the signal waveform by converting the plurality of units into a predetermined common unit.

### Supplementary Note 6

The diagnosis support system according to any one of supplementary notes 1 to 5, further including
an input unit for, in a case where a diagnostic image including signal waveforms of a plurality of types of biological signals is input for the same user, outputting a diagnostic result of medical diagnosis with respect to the user, and inputting a diagnostic image including the signal waveform whose scale has been adjusted by the adjustment unit to a learned model learned by a predetermined machine learning algorithm, and
an output unit for outputting the diagnostic result for the diagnostic image input by the input unit from the learned model.

### Supplementary Note 7

The diagnosis support system according to any one of supplementary notes 1 to 6, further including a generation unit for generating a diagnostic image including the signal waveform whose scale has been adjusted,
in which the generation unit generates the diagnostic image by superimposing signal waveforms of the plurality of types of biological signals whose scales have been adjusted.

### Supplementary Note 8

The diagnosis support system according to supplementary note 7, in which the generation unit generates the diagnostic image based on a layout defined in advance for the arrangement of the adjusted signal waveforms.

### Supplementary Note 9

The diagnosis support system according to any one of supplementary notes 1 to 8, in which the adjustment unit adjusts a scale of the biological signal axis by expanding the signal waveform in the original image in a biological signal axis direction in the signal waveform.

### Supplementary Note 10

The diagnosis support system according to any one of supplementary notes 2 to 5, in which the adjustment unit adjusts an adjustment degree of the scale of the time axis to be larger than an adjustment degree of the scale of the biological signal axis.

### Supplementary Note 11

The diagnosis support system according to supplementary note 10, in which the adjustment unit adjusts only the scale of the time axis.

### Supplementary Note 12

A diagnosis support method in which a computer supports a medical diagnosis for a user, the diagnosis support method including
acquiring an original image including signal waveforms of a plurality of types of biological signals for the same user, and
adjusting at least one of scales of the plurality of signal waveforms in the original image based on a predetermined adjustment criterion.

### Supplementary Note 13

A program for supporting medical diagnosis for a user, the program causing a computer to execute
acquiring an original image including signal waveforms of a plurality of types of biological signals for the same user, and
adjusting at least one of scales of a plurality of the signal waveforms in the original image based on a predetermined adjustment criterion.

Some or all of the elements (e.g., configurations and functions) described in supplementary notes 2 to 11 dependent on supplementary note 1 can also depend on supplementary notes 12 and 13 by the dependency relationships similar to those of supplementary notes 2 to 11. Some or all of the elements described in any supplementary note can be applied to various types of hardware, software, recording means for recording software, systems, and methods.

1. A diagnosis support system that supports a medical diagnosis for a user, the diagnosis support system comprising:
at least one memory storing instructions, and
at least one processor configured to execute the instructions to;
acquire an original image including signal waveforms of a plurality of types of biological signals for the same user; and
adjust at least one of scales of the plurality of signal waveforms in the original image based on a predetermined adjustment criterion.

2. The diagnosis support system according to claim 1, wherein the at least one processor is further configured to execute the instructions to adjust at least one of a scale of a time axis and a scale of a biological signal axis in the signal waveform independently of the other scale.

3. The diagnosis support system according to claim 1, wherein the at least one processor is further configured to execute the instructions to adjust the scale based on normal biological data for the biological signal.

4. The diagnosis support system according to claim 3, wherein the at least one processor is further configured to execute the instructions to:
acquire an attribute of the user; and
adjust the scale based on the normal biological data in the attribute.

5. The diagnosis support system according to claim 1, wherein
the plurality of types of biological signals includes a plurality of units, and
the at least one processor is further configured to execute the instructions to adjust a scale of a biological signal axis in the signal waveform by converting the plurality of units into a predetermined common unit.

6. The diagnosis support system according to claim 1, wherein the at least one processor is further configured to execute the instructions to;
input a diagnostic image including the signal waveform whose scale has been adjusted to a learned model learned by a predetermined machine learning algorithm, wherein the learned model outputs a diagnostic result of medical diagnosis with respect to a user, in a case where a diagnostic image including signal waveforms of a plurality of types of biological signals is input for the same user; and
output the diagnostic result for the diagnostic image input from the learned model.

7. The diagnosis support system according to claim 1, wherein the at least one processor is further configured to execute the instructions to generate a diagnostic image including the signal waveform whose scale has been adjusted, by superimposing signal waveforms of the plurality of types of biological signals whose scales have been adjusted.

8. The diagnosis support system according to claim 7, wherein the at least one processor is further configured to execute the instructions to generate the diagnostic image based on a layout defined in advance for the arrangement of the adjusted signal waveforms.

9. The diagnosis support system according to claim 1, wherein the at least one processor is further configured to execute the instructions to adjust a scale of the biological signal axis by expanding the signal waveform in the original image in a biological signal axis direction in the signal waveform.

10. The diagnosis support system according to claim 2, wherein the at least one processor is further configured to execute the instructions to adjust an adjustment degree of the scale of the time axis to be larger than an adjustment degree of the scale of the biological signal axis.

11. The diagnosis support system according to claim 10, wherein the at least one processor is further configured to execute the instructions to adjust only the scale of the time axis.

12. A diagnosis support method in which a computer supports a medical diagnosis for a user, the diagnosis support method comprising:
acquiring an original image including signal waveforms of a plurality of types of biological signals for the same user; and
adjusting at least one of scales of the plurality of signal waveforms in the original image based on a predetermined adjustment criterion.

13. A non-transitory computer-readable medium storing a program for supporting medical diagnosis for a user, the program causing a computer to execute:
acquiring an original image including signal waveforms of a plurality of types of biological signals for the same user; and
adjusting at least one of scales of a plurality of the signal waveforms in the original image based on a predetermined adjustment criterion.
